(19) United States
(12) Patent Application Publication
(10) Pub. No.: US 2019/0343128 A1
(43) Pub. Date: Nov. 14, 2019

(54) DO IT YOURSELF CANNABIS BAKING KIT

(71) Applicant: Tony Sary, Newport Beach, CA (US)

(72) Inventor: Tony Sary, Newport Beach, CA (US)

(21) Appl. No.: 15/974,608

(22) Filed: May 8, 2018

(51) Int. Cl. A21B 3/13; A23L 33/105; A47J 37/01; A47J 43/07; A61K 36/185; G01F 23/02

(52) U.S. Cl. CPC A21B 3/13; A23L 33/105; G01F 23/02; A47J 43/0727; A61K 36/185; A47J 37/01

(57) ABSTRACT

An apparatus for preparing a cannabis edible, including methods of delivery and preparation, comprises a first container, a second container, and a pan which are secured in a first housing. The apparatus further includes a tray which together with the first housing is placed in a second housing. The first container includes a lid and is used to store cannabis extract having a predetermined dosage of cannabinoid resin. The second container is used to store a food mix. The pan is used to mix the cannabis extract and the food mix in a mixture. The tray is used to cover an open top section of the pan after the mixture is heated and may further include a plurality of notches along its sides as portion indicators.

### COPYRIGHT

A portion of the disclosure of this patent document contains material which is subject to copyright protection. The owner has no objection to the facsimile reproduction by anyone of the patent disclosure, as it appears in the Patent and Trademark Office files or records, but otherwise reserves all copyright rights whatsoever.

### FIELD OF INVENTION

The present invention relates to an apparatus for preparing a cannabis edible and methods of delivery and preparation of the cannabis edible. In particular, a first container includes a lid and is used to store cannabis extract having a predetermined dosage of cannabinoid resin. A second container is used to store a food mix, and a pan is used to mix the cannabis extract and the food mix in a mixture which may include other ingredients, such as water. A tray is provided to cover the pan after the mixture is heated and a plurality of notches along at least one side of the tray may be used to cut the edible into portions. A first housing is used to secure the first container, the second container and the pan, and a second housing is used to store the first housing and the tray.

### BACKGROUND

Cannabis-infused foods, commonly referred to as edibles, are a popular choice for use of cannabis. Edibles are particularly desirable by those who would like to receive the benefits of cannabis without the harm that is caused as a result of smoking it or otherwise making it come into contact with the lungs. However, edibles have disadvantages associated with their use. Specifically, edibles must be consumed in a short period of time before they become stale. Furthermore, because they are digested, the amount of cannabis in the edibles must be accurately measured or they can cause overdose. Therefore, there is a need to provide an apparatus which can be used to deliver the ingredients of a cannabis edible so that accurately measured amount of it can be prepared fresh at anytime and anyplace.

### SUMMARY

In one aspect, an apparatus for preparing a cannabis edible is disclosed wherein the apparatus comprises a first container having a lid, said first container comprising cannabis extract having a predetermined dosage of cannabinoid resin, a second container comprising a food mix, a pan for mixing the cannabis extract and the food mix in a mixture therein, wherein the pan is operable to heat the mixture when said pan is placed in an oven, a tray operable to contact a top rim of the pan, thereby, covering an open top section of the pan, a first housing operable to receive the first container, the second container, and the pan and a second housing operable to receive the first housing and the tray.

Preferably, the first container is made from one of plexiglass and glass.

Preferably, the first container includes measurement indicators on a wall of said first container operable to enable the first container to be used as a measuring container for inclusion of other ingredients in the mixture.

Preferably, the first container is hermetically sealed via tamper-resistant induction sealing.

Preferably, the cannabinoid resin is at least one of tetrahydrocannabinol (THC) oil and cannabidiol (CBD) oil.

Preferably, the second container comprises two or more compartments for the food mix.

Preferably, the food mix comprises at least one of fresh food products and processed food products. Preferably, the fresh food products comprise at least one of popcorn seeds, fresh vegetables, fresh meats, and fresh poultry. Preferably, the processed food products comprise at least one of a cookie mix, a cake mix, a brownie mix, and a bread mix.

Preferably, the first housing comprises a first cavity to receive the first container, a second cavity to receive the second container, and a third cavity to receive the pan.

Preferably, the tray comprises a plurality of notches along at least one of a first side and a second side of said tray, said notches operable to be used as portion indicators.

Preferably, the pan is microwave-safe.

Preferably, the first housing is made from a paper-based material.

Preferably, the second container is made from a paper-based material.

Preferably, the second housing is made from a paper-based material.

Preferably, the second housing includes cooking instructions and nutritional facts.

In another aspect, a method of delivering ingredients of a cannabis edible is disclosed wherein the method comprises providing a first container having a lid, said first container comprising cannabis extract having a predetermined dosage of cannabinoid resin, providing a second container comprising a food mix, providing a pan for mixing the cannabis extract and the food mix in a mixture therein, wherein the pan is operable to heat the mixture when said pan is placed in an oven, providing a tray operable to contact a top rim of the pan, thereby, covering an open top section of the pan, providing a first housing operable to receive the first container, the second container, and the pan, and providing a second housing operable to receive the first housing and the tray.

In another aspect, a method of preparing a cannabis edible is disclosed wherein the method comprises mixing a cannabis extract and a food mix for making a mixture in a pan, heating the mixture by placing the pan in an oven, removing the pan from the oven, and covering an open top section of the pan via a tray operable to contact a top rim of the pan, wherein the cannabis extract has a predetermined dosage of cannabinoid resin, said extract is contained in a first container having a lid, wherein the food mix is contained in a second container, wherein the first container, the second container, and the pan are housed within a first housing operable to receive the first container, the second container, and the pan, and wherein the first housing and the tray are housed within a second housing operable to receive the first housing and the tray.

Preferably, the method further comprises mixing one or more ingredients in the mixture.

Preferably, the one or more ingredients comprises water.

### DESCRIPTION OF THE DRAWINGS

FIG. 1 shows a perspective exploded view of a preferred embodiment of an apparatus for preparing a cannabis edible comprising a first container, a second container, and a pan, secured within a first housing, and a tray placed beneath the first housing and together stored in a second housing according to the present invention.

FIG. 2 shows a perspective exploded view of a first housing, such as the first housing of FIG. 1, showing a first container, a second container, a pan, and a tray.

FIG. 3 shows a front view of an unfolded second housing, such as the second housing of FIG. 1, showing a plurality of perforations, used to fold the second housing, and two regions where cooking instructions and nutritional facts may be provided.

FIG. 4 shows a top view of an unfolded first housing, such as the first housing of FIG. 1, showing a plurality of perforations, used to fold the first housing, a first cavity and a second cavity, used to receive a first container and a second container.

FIG. 5 shows a top view of a tray, such as the tray of FIG. 1, showing a plurality of notches along two sides of the tray, used as portion indicators.

FIG. 6A shows a side view of a pan and a first container, such as the pan and the first container of FIG. 1, showing cannabis extract having a predetermined dosage of cannabinoid resin inside the first container being poured into the pan to prepare a cannabis edible.

FIG. 6B shows a side view of the pan and the first container of FIG. 6A, showing the first container being used to add a measured amount of water being poured into the pan.

FIG. 6C shows a side view of the pan of FIG. 6A and a second container, such as the second container of FIG. 1, showing a food mix being added into the pan.

FIG. 6D shows a side view of the pan of FIG. 6C, showing a mixture made from mixing the cannabis extract, the food mix, and the water inside the pan.

FIG. 6E shows a side view of the pan of FIG. 6D inside an oven to heat the mixture inside the pan.

FIG. 6F shows a side view of the pan of FIG. 6E after being removed from the oven, showing a tray, such as the tray of FIG. 1, coming into contact with a top rim of the pan in order to cover an open top section of the pan.

FIG. 6G shows a side view of the pan of FIG. 6F after being inverted and the pan removed, showing the cannabis edible on the tray of FIG. 6F.

FIG. 6H shows a side view of the cannabis edible on the tray of FIG. 6G after the cannabis edible has been portioned utilizing the notches of the tray as portion indicators.

### DETAILED DESCRIPTION OF THE PRESENTLY PREFERRED EMBODIMENTS

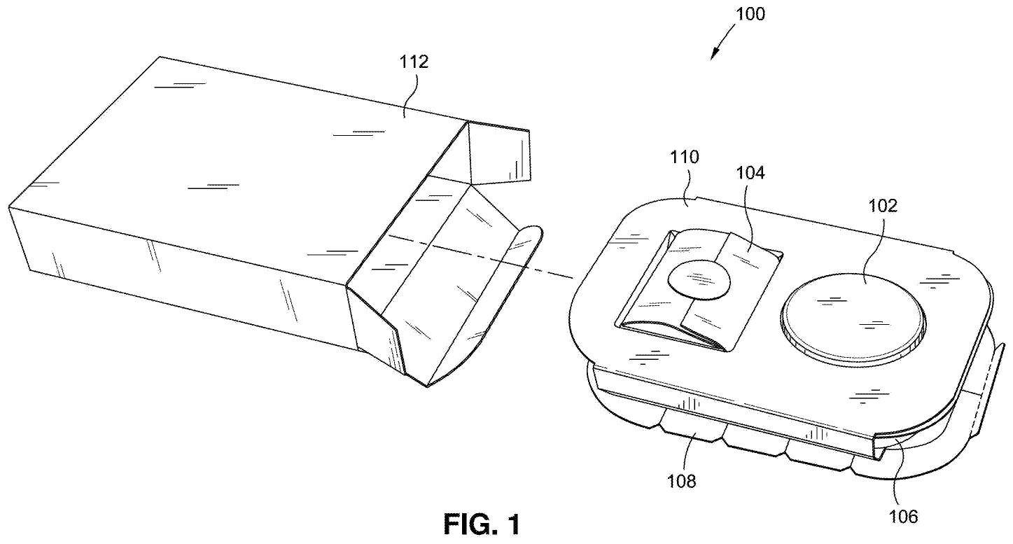

FIG. 1 depicts a perspective exploded view of a preferred embodiment of an apparatus for preparing a cannabis edible 100 according to the present invention. The apparatus 100 comprises a first container 102, a second container 104, and a pan 106 which are secured within a first housing 110. A tray 108 is also included which is used to place and portion the edible, discussed in more detail below, after the edible has been prepared. The tray 108 is placed beneath the first housing 110 and together inserted inside a second housing 112 which is subsequently closed and ready for delivery. As used herein, the terms container, pan, and housing are interchangeable.

The first container 102 contains an accurately measured amount of cannabis extract, having a predetermined dosage of cannabinoid resin. In a preferred embodiment, the cannabinoid resin is a cannabis oil. Illustrative examples of the cannabis oil are the tetrahydrocannabinol (THC) oil and/or cannabidiol (CBD) oil. The second container 104 contains a food mix. The food mix may comprise fresh food, processed food, or a combination thereof. Illustrative examples of the fresh food are popcorn seeds, fresh vegetables, fresh meats, and fresh poultry. Illustrative examples of the process food are cookie mix, cake mix, brownie mix, and bread mix.

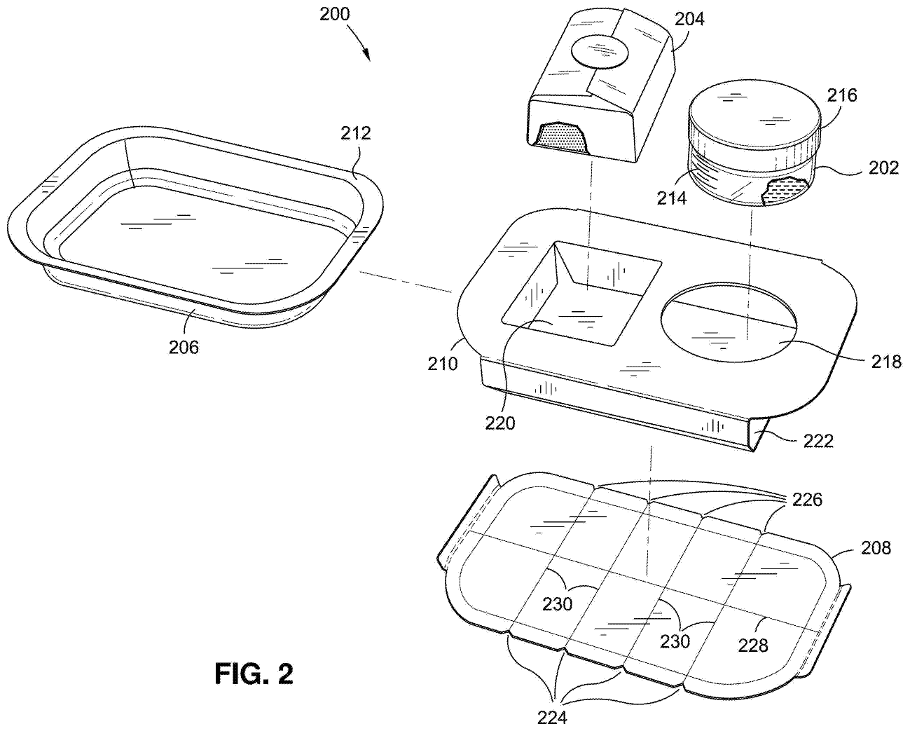

FIG. 2 depicts a perspective exploded view 200 of a first housing 210, a first container 202, a second container 204, a pan 206, and a tray 208. The first container 202 includes measurement indicators 214 on a wall of said first container 202 which enables the first container 202 to be used as a measuring container for inclusion of other ingredients such as water. The first container 202 further includes a lid 216. In a preferred embodiment, the lid 216 is a child-resistant lid. In a preferred embodiment the lid 216 is coupled to the first container 202 so as to make the first container 202 hermetically sealed. An illustrative example of method of hermetically sealing the container 202 is tamper-resistant induction sealing.

In a preferred embodiment the first container 202 is made from plexiglass. In another preferred embodiment, the first container 202 is made from glass. The first container 202 may have different shapes. In a preferred embodiment, the first container 202 has cylindrical shape.

The second container 204 contains a food mix. In a preferred embodiment, the second container 204 comprises two or more compartments where different ingredients may be stored. In a preferred embodiment, the second container 204 is made from a paper-based material such as cardboard. In another preferred embodiment, the second container 204 is made from plastic. Different shapes for the second container 204 are contemplated to include rectangular as well as circular.

The pan 206 includes a top rim 212 which comes into contact with the tray 208. In a preferred embodiment, the pan 206 is made from a microwave-safe material. After a mixture is made within the pan 206 and heated to cook the edible, for instance inside a gas oven or a microwave oven, the tray 208 is placed on top of the pan 206 making contact with the top rim 212, thereby, covering the edible inside the pan 206.

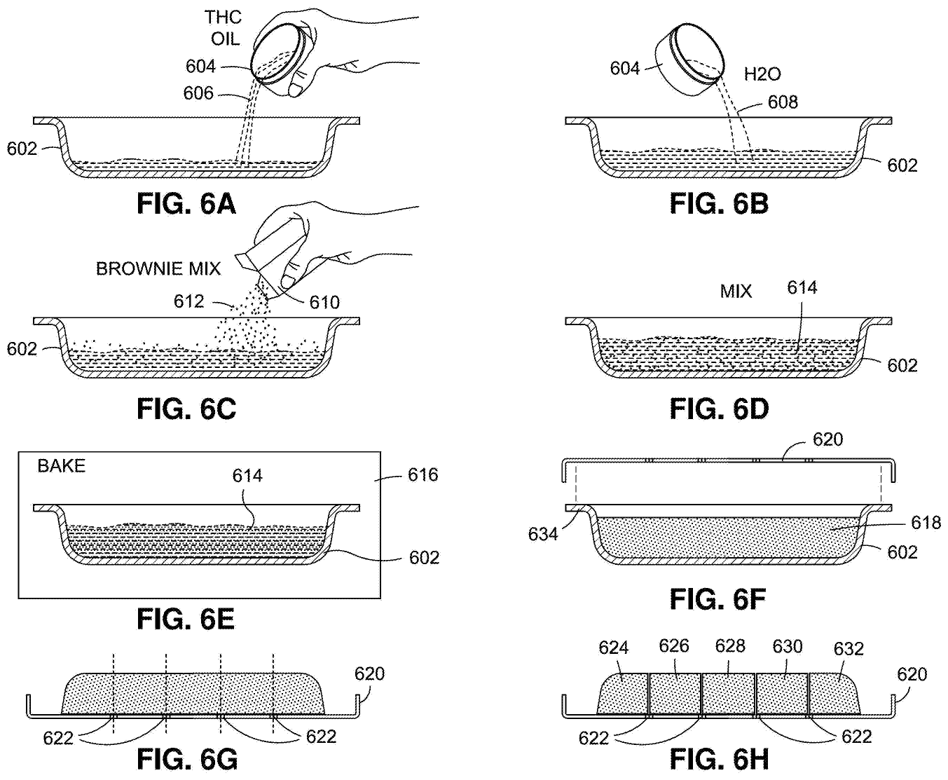

The tray 208 is used to place and cut the edible into portions after it has been prepared. In a preferred embodiment, the tray 208 includes a plurality of notches 224 along one of its sides and another plurality of notches 226 along the other side to be used as portion indicators (shown in FIGS. 6G and 6H). In a preferred embodiment, the tray 208 further includes lines 228 and 230 which have been drawn on the side of the tray 208 where the edible is placed. The lines 228 and 230 may be further utilized as portion indicators to cut the edible into portions for servings.

The first housing 210 is used to receive the first container 202, the second container 204, and the pan 206. In a preferred embodiment, the first housing 210 comprises a first cavity 218 to receive the first container 202, a second cavity 220 to receive the second container 204, and a third cavity 222 to receive the pan 206. In a preferred embodiment, the first housing 210 is made from a paper-based material.

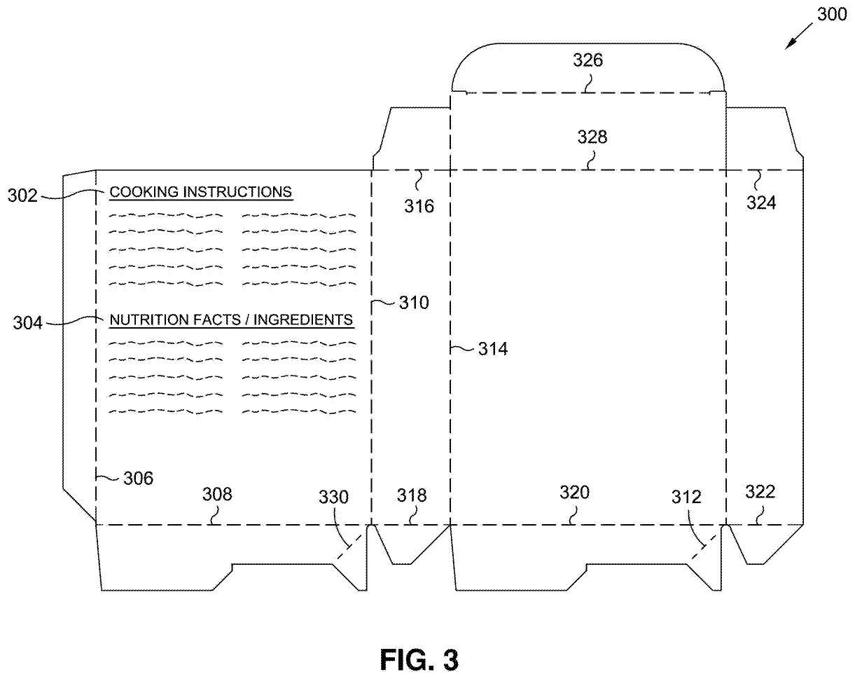

FIG. 3 depicts a front view of an unfolded second housing 300. In this preferred embodiment, the second housing 300 is made from a paper-based material. The housing 300 includes a plurality of perforations, 306, 308, 310, 312, 314, 316, 318, 320, 322, 324, 326, 328, and 330, used to fold the second housing 300 into a container. In a preferred embodiment, the second housing 300 includes two regions, 302 and 304, where cooking instructions and nutritional facts may be provided, respectively.

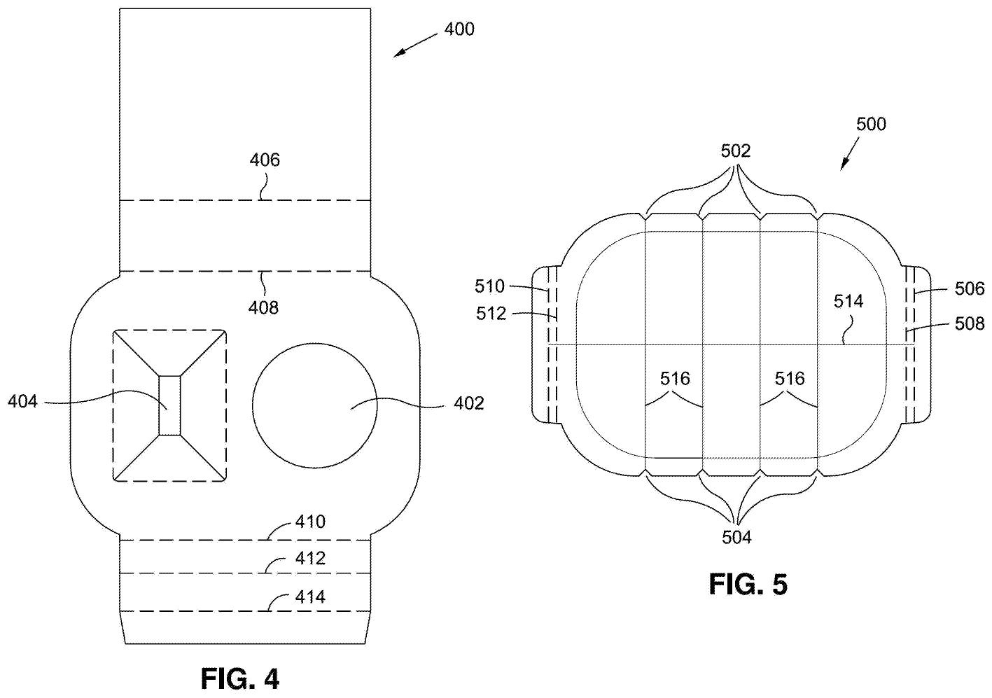

FIG. 4 depicts a top view of an unfolded first housing 400. In this preferred embodiment, the first housing 400 is made from a paper-based material. The first housing 400 includes a plurality of perforations 406, 408, 410, 412, and 414 which can be used to fold the first housing 400. A first cavity 402, a second cavity 404, and a third cavity such as the cavity 222 shown in FIG. 2, will be created upon folding the first housing 400 where they can be used to receive a first container a second container, and a pan, such as the first container 202, the second container 204, and the pan 206 of FIG. 2.

FIG. 5 depicts a top view of a tray 500. In this preferred embodiment, the tray 500 includes a plurality of notches 502 and 504 along two sides of the tray 500. As discussed above, these notches may be used as portion indicators. The tray 500 may be made from a paper-based material which includes a plurality of perforations 506, 508, 510, and 512. In addition, lines 514 and 516 can be drawn on the tray 500 to further allow one to portion the edible after it has been placed on the tray 500 (see FIGS. 6G and 6H.)

FIGS. 6A through 6H illustrate a method of preparing a cannabis edible utilizing the apparatus 100. FIG. 6A depicts a side view of a pan 602 and a first container 604. Cannabis extract 606 having a predetermined dosage of cannabinoid resin inside the first container 604 is poured into the pan 602 to prepare the cannabis edible. FIG. 6B depicts a side view of the pan 602 and the first container 604, the latter being used to add a measured amount of water 608 into the pan 602. The first container 602 includes measurement indicators which can be used to add additional ingredients into the mixture. FIG. 6C depicts a side view of the pan 602 and a second container 610. The second container 610 contains a food mix 612 which can be added into the pan 620 to mix with the mixture. Specifically, FIG. 6D depicts a side view of the pan 602 after a mixture 614 is made by mixing the cannabis extract 606, the food mix 612, and the water 608 inside the pan 602. FIG. 6E depicts a side view of the pan 602 after it has been placed inside an oven 616 to heat the mixture 614 inside the pan 602. FIG. 6F depicts a side view of the pan 602 after being removed from the oven 616. A tray 620 is brought into coming into contact with a top rim 634 of the pan 602 in order to cover an open top section of the pan 602. FIG. 6G depicts a side view of the pan 602 which has been inverted. The pan 602 is then removed, exposing the cannabis edible 618 on the tray 620. Side view of a plurality of notches 622 can be seen. FIG. 6H depicts a side view of the cannabis edible 618 on the tray 620 after it has cut into portions 624, 626, 628, 630, and 632 utilizing the notches 622 of the tray 620.

The foregoing explanations, descriptions, illustrations, examples, and discussions have been set forth to assist the reader with understanding this invention and further to demonstrate the utility and novelty of it and are by no means restrictive of the scope of the invention. It is the following claims, including all equivalents, which are intended to define the scope of this invention.

What is claimed is:

1. An apparatus for preparing a cannabis edible, comprising:
(a) a first container having a lid, said first container comprising cannabis extract having a predetermined dosage of cannabinoid resin;
(b) a second container comprising a food mix;
(c) a pan for mixing the cannabis extract and the food mix in a mixture therein, wherein the pan is operable to heat the mixture when said pan is placed in an oven;
(d) a tray operable to contact a top rim of the pan, thereby, covering an open top section of the pan;
(e) a first housing operable to receive the first container, the second container, and the pan; and
(f) a second housing operable to receive the first housing and the tray.

2. The apparatus of claim 1, wherein the first container is made from one of plexiglass and glass.

3. The apparatus of claim 1, wherein the first container includes measurement indicators on a wall of said first container operable to enable the first container to be used as a measuring container for inclusion of other ingredients in the mixture.

4. The apparatus of claim 1, wherein the first container is hermetically sealed via tamper-resistant induction sealing.

5. The apparatus of claim 1, wherein the cannabinoid resin is at least one of tetrahydrocannabinol (THC) oil and cannabidiol (CBD) oil.

6. The apparatus of claim 1, wherein the second container comprises two or more compartments for the food mix.

7. The apparatus of claim 1, wherein the food mix comprises at least one of fresh food products and processed food products.

8. The apparatus of claim 7, wherein the fresh food products comprise at least one of popcorn seeds, fresh vegetables, fresh meats, and fresh poultry.

9. The apparatus of claim 7, wherein the processed food products comprise at least one of a cookie mix, a cake mix, a brownie mix, and a bread mix.

10. The apparatus of claim 1, wherein the first housing comprises a first cavity to receive the first container, a second cavity to receive the second container, and a third cavity to receive the pan.

11. The apparatus of claim 1, wherein the tray comprises a plurality of notches along at least one of a first side and a second side of said tray, said notches operable to be used as portion indicators.

12. The apparatus of claim 1, wherein the pan is microwave-safe.

13. The apparatus of claim 1, wherein the first housing is made from a paper-based material.

14. The apparatus of claim 1, wherein the second container is made from a paper-based material.

15. The apparatus of claim 1, wherein the second housing is made from a paper-based material.

16. The apparatus of claim 1, wherein the second housing includes cooking instructions and nutritional facts.

17. A method of delivering ingredients of a cannabis edible, comprising:
(a) providing a first container having a lid, said first container comprising cannabis extract having a predetermined dosage of cannabinoid resin;
(b) providing a second container comprising a food mix;
(c) providing a pan for mixing the cannabis extract and the food mix in a mixture therein, wherein the pan is operable to heat the mixture when said pan is placed in an oven;
(d) providing a tray operable to contact a top rim of the pan, thereby, covering an open top section of the pan;
(e) providing a first housing operable to receive the first container, the second container, and the pan; and
(f) providing a second housing operable to receive the first housing and the tray.

18. A method of preparing a cannabis edible, comprising:
(a) mixing a cannabis extract and a food mix for making a mixture in a pan;
(b) heating the mixture by placing the pan in an oven;
(c) removing the pan from the oven; and
(d) covering an open top section of the pan via a tray operable to contact a top rim of the pan;
wherein the cannabis extract has a predetermined dosage of cannabinoid resin, said extract is contained in a first container having a lid;
wherein the food mix is contained in a second container;
wherein the first container, the second container, and the pan are housed within a first housing operable to receive the first container, the second container, and the pan; and
wherein the first housing and the tray are housed within a second housing operable to receive the first housing and the tray.

19. The method of claim 18, further mixing one or more ingredients in the mixture.

20. The method of claim 19, wherein the one or more ingredients comprises water.
